(19) United States
(12) Patent Application Publication
(10) Pub. No.: US 2016/0376544 A1
(43) Pub. Date: Dec. 29, 2016

(54) PROCESS FOR ENRICHMENT OF MICROALGAL BIOMASS WITH CAROTENOIDS AND WITH PROTEINS

(71) Applicant: ROQUETTE FRERES, Letrem (FR)

(72) Inventors: MATHIEU COSSART, MARLES LES MINES (FR); SOPHIE DEFRETIN, BETHUNE (FR); GABRIEL MACQUART, MONT BERNANCHON (FR); LAURENT SEGUEILHA, MARQUETTE LEZ LILLE (FR)

(21) Appl. No.: 15/039,428

(22) PCT Filed: Nov. 28, 2014
(86) PCT No.: PCT/FR2014/053075

§ 371 Date: May 26, 2016

(30) Foreign Application Priority Data
Nov. 29, 2013 (FR) 1361837

(51) Int. Cl. C12N 1/12; C12P 21/00; C12P 23/00

(52) U.S. Cl. CPC C12N 1/12; C12P 23/00; C12P 21/00

(57) ABSTRACT

The invention relates to a process for the enrichment, with carotenoids and proteins, of a biomass of a microalga cultivated under heterotrophic conditions, wherein said microalga is of the Chlorella genus, which comprises culturing said microalga in a minimum medium supplemented with a nitrogen source in organic form, preferably chosen from the group consisting of yeast extract, corn steep liquor, and a combination thereof.

The present invention relates to a method for carotenoid enrichment and protein enrichment of a microalgal biomass, more particularly of the Chlorella genus, more particularly still of the species Chlorella sorokiniana.

Macroalgae and microalgae have a specific richness which remains largely unexplored. Their utilization for dietary, chemical or bioenergy purposes is still highly marginal. Nonetheless, they contain components of great value.

Indeed, microalgae are sources of vitamins, lipids, proteins, sugars, pigments and antioxidants.

Algae and microalgae are thus of interest to the industrial sector, where they are used for manufacturing food supplements, functional foods, cosmetics, medication or for aquaculture.

Microalgae are first and foremost photosynthetic microorganisms which colonize all biotopes exposed to light.

On the industrial scale, the monoclonal culturing thereof is carried out in photobioreactors (autotrophic conditions: in light with CO₂) or, for some, it is also carried out in fermenters (heterotrophic conditions: in darkness in the presence of a source of carbon).

This is because some species of microalgae are able to grow in the absence of light: Chlorella, Nitzschia, Cyclotella, Tetraselmis, Crypthecodinium, Schizochytrium.

Moreover, it is estimated that culturing in heterotrophic conditions is 10 times less expensive than in phototrophic conditions because, for those skilled in the art, these heterotrophic conditions allow:
the use of fermenters identical to those used for bacteria and yeast, enabling all the culturing parameters to be controlled, and
the production of biomasses in much greater amounts than those obtained by light-based culturing.

The profitable utilization of microalgae generally necessitates controlling the fermentation conditions, making it possible to accumulate their components of interest, such as:
pigments (chlorophyll a, b and c, β-carotene, astaxanthin, lutein, phycocyanin, xanthophylls, phycoerythrin, etc), the demand for which is increasing both due to their noteworthy antioxidant properties and to their provision of natural colorings for food,
proteins, in order to optimize the nutritional qualities thereof, or
lipids, in order to optimize their content of fatty acids (up to 60%, or even 80% by weight of their solids), especially for:
   biofuel applications, but also
   applications in food for human consumption or animal feed, when the chosen microalgae produce “essential” (i.e. supplied by the diet because they are not naturally produced by humans or animals) polyunsaturated fatty acids or PUFAs.

To achieve this result, first methods for fermentation making it possible to obtain high cell densities (HCD) have thus been thoroughly investigated in order to obtain maximum protein or lipid yields and productivity.

The aim of these HCD cultures was to obtain the highest possible concentration of the desired product in the shortest possible period of time.

This principle is borne out for example by the biosynthesis of astaxanthin by Chlorella zofingiensis, in which growth of the microalga has proved to be directly correlated with the production of this compound (Wang and Peng, 2008, World J Microbiol. Biotechnol., 24(9), 1915-1922).

However, maintaining growth at its maximum rate (μ, in h⁻¹) is not always correlated with high production of the desired product.

Indeed, it quickly became apparent to specialists in the field that it is necessary, for example, to subject the microalgae to a nutritional stress which limits their growth, when it is desired to make them produce large lipid stores.

Therefore, in fermenting methods, growth and production are henceforth uncoupled.

For example, to promote the accumulation of polyunsaturated fatty acids (in this instance docosahexanoic acid or DHA), patent application WO 01/54510 recommends dissociating cell growth from the production of polyunsaturated fatty acids.

In the microalga Schizochytrium sp., strain ATCC 20888, a first growth phase is thus carried out without limiting oxygen, so as to promote obtaining a high cell density (more than 100 g/l), then, in a second phase, the supply of oxygen is gradually slowed so as to stress the microalga, slow its growth and trigger production of the fatty acids of interest.

In the microalga Crypthecodinium cohnii, the highest content of docosahexanoic acid (DHA, a polyunsaturated fatty acid) is obtained at low glucose concentration (of the order of 5 g/l) and thus at a low growth rate (Jiang and Chen, 2000, Process Biochem., 35(10), 1205-1209).

These results are a good illustration of the fact that the product formation kinetics can be associated both positively and negatively with growth of the microalgae, or even a combination of the two.

Consequently, in the event that the formation of products is not correlated with high cell growth, it is prudent to control the rate of cell growth.

In general, those skilled in the art choose to control the growth of the microalgae by controlling the fermentation conditions (temp, pH) or by regulated feeding of nutritional components to the fermentation medium (semi-continuous conditions referred to as “fed batch”).

If they choose to control the growth of the microalgae in heterotrophy through the supply of carbon sources, those skilled in the art generally choose to adapt the carbon source (pure glucose, acetate, ethanol, etc.) to the microalga (C. cohnii, Euglena gracilis, etc.) as a function of the metabolite produced (for example a polyunsaturated fatty acid of DHA type).

Temperature may also be a key parameter:
for example, it has been reported that the synthesis of polyunsaturated fatty acids in some species of microalgae, such as EPA by Chlorella minutissima, is promoted at a lower temperature than that required for the optimal growth of said microalga;
on the other hand, the lutein yield is higher in heterotrophically cultivated Chlorella protothecoides when the production temperature is increased from 24 to 35° C.

Indeed, Chlorella protothecoides is acknowledged to be one of the best oil-producing microalgae.

In heterotrophic conditions, it rapidly converts carbohydrates to triglycerides (more than 50% of the solids thereof).

To optimize this production of triglycerides, those skilled in the art are led to optimize the carbon flow toward oil production, by acting on the nutritional environment of the fermentation medium.

Thus, it is known that oil accumulates when there is a sufficient supply of carbon but under conditions of nitrogen deficiency.

Therefore, the C/N ratio is the determining factor here, and it is accepted that the best results are obtained by acting directly on the nitrogen content, with the glucose content not being a limiting factor.

Unsurprisingly, this nitrogen deficiency affects cell growth, which results in a growth rate 30% lower than the normal growth rate for the microalga (Xiong et al., Plant Physiology, 2010, 154, pp. 1001-1011).

To explain this result, in the abovementioned article Xiong et al. in fact demonstrate that if the Chlorella biomass is divided into its 5 main components, i.e. carbohydrates, lipids, proteins, DNA and RNA (representing 85% of the solids thereof), while the C/N ratio has no impact on the content of DNA, RNA or carbohydrates, it becomes paramount for the content of proteins and lipids.

Thus, Chlorella cells cultivated with a low C/N ratio contain 25.8% proteins and 25.23% lipids, whereas a high C/N ratio makes the synthesis of 53.8% lipids and 10.5% proteins possible.

To optimize its oil production, it is therefore essential for those skilled in the art to control the carbon flow by steering it toward oil production to the detriment of protein production; the carbon flow is redistributed and accumulates as lipid storage substances when the microalgae are placed in a nitrogen-deficient medium.

Armed with this teaching, in order to produce protein-rich biomasses, those skilled in the art are therefore led to perform the opposite of this metabolic control, i.e. to modify the fermentation conditions by instead promoting a low C/N ratio, and thus:
supply a large amount of nitrogen source to the fermentation medium while keeping constant the carbon source feedstock, which will be converted into proteins, and
stimulate the growth of the microalga.

This involves modifying the carbon flow toward protein (and hence biomass) production, to the detriment of storage lipid production.

Within the context of the invention, the applicant company has chosen to explore an original route by proposing an alternative solution to that conventionally envisioned by those skilled in the art.

Thus, the invention relates to a method for carotenoid enrichment and protein enrichment of a heterotrophically cultivated microalgal biomass, said microalga being of the Chlorella genus, more particularly still Chlorella sorokiniana, which heterotrophic culturing method comprises culturing said microalga in a minimum medium supplemented with a nitrogen source chosen from the group consisting of a yeast extract and a corn steep liquor, and a combination thereof.

Within the context of the invention,
carotenoid “enrichment” is intended to mean that the content of carotenoids in the biomass is increased by at least 0.05%, preferably by at least 0.1% by total weight of the biomass, compared to the content of carotenoids in the biomass cultivated solely in minimum medium. Preferably, the biomass obtained by the method according to the invention has a content of carotenoids of at least 0.35% by total weight of the biomass, more particularly preferably at least 0.4% by total weight of the biomass;
protein “enrichment” is intended to mean that the content of proteins in the biomass is increased by at least 5%, preferably by at least 10% by total weight of the biomass, compared to the content of proteins in the biomass cultivated solely in minimum medium. Preferably, the biomass obtained by the method according to the invention has a content of proteins of at least 45% by total weight of the biomass, more particularly preferably at least 50% by total weight of the biomass.

Within the context of the invention, “minimum medium” is conventionally defined as a medium which only contains those chemical elements strictly necessary to the growth of the microalga, in a form which can be used by microalgae not having any specific requirements.

The minimum medium therefore contains:
a source of carbon and of energy: generally glucose
a source of potassium and of phosphorus: for example K₂HPO₄
a source of nitrogen and of sulfur: for example (NH₄)₂SO₄
a source of magnesium: for example MgSO₄.7H₂O
a source of calcium: for example CaCl₂.2H₂O
a source of iron: for example FeSO₄.7H₂O
sources of trace elements: salts of Cu, Zn, Co, B, Mn, Mo
sources of vitamins (thiamine, biotin, vitamin B12, etc).

The applicant company then found that, while culturing a microalga of the Chlorella genus, more particularly still Chlorella sorokiniana, in this essentially inorganic minimum medium still made it possible to produce a large amount of biomass, this was to the detriment of the components of interest such as carotenoids and proteins.

The high growth rate of the biomass (more than 0.05 h⁻¹) in essentially inorganic medium reflects notably the autotrophy of the strain with respect to nitrogen.

Without being bound by any theory, the applicant company then put forward the hypothesis that culturing microalgae in minimum medium steered the metabolic pathways toward the production of storage substances (of polysaccharide type).

The applicant company then found that supplying a small amount of a nitrogen-based nutritional supplement in an organic form (the nitrogen supply preferably remains more than 90% inorganic), that is to say in the form of yeast extracts or corn steep liquor (CSL), in these specific conditions (while the microalga is completely autotrophic for nitrogen) made it possible to slow the production of said polysaccharide storage substances and to divert the metabolic pathways toward carotenoid and protein production.

Carrying out the fermentation in this way thus makes it possible to easily manage, in the minimum medium, the addition of the nitrogen-based nutritional supplements which increase the production of carotenoids and proteins.

This strategy therefore goes heavily against the technical preconception that, for example, to increase the content of proteins in the biomass, it is absolutely imperative to increase this biomass and therefore the cell growth, or to boost the fermentation medium with sources of nitrogen.

Indeed, the amount of biomass here remains constant, and the addition of the nutritional supplements (preferably less than 10% by weight of the total nitrogen added to the fermentation medium) is what leads to overproduction of proteins.

Thus, the present invention relates to a method for carotenoid enrichment and protein enrichment of a heterotrophically cultivated microalgal biomass, said heterotrophic culturing method comprising culturing said microalga in a minimum medium supplemented with a nitrogen source in organic form.

The microalga is preferably of the Chlorella genus, in particular a pigment-rich microalga chosen from Chlorella sorokiniana, Chlorella vulgaris and Chorella kessleri, and more particularly preferably Chlorella sorokiniana.

Preferably, the nitrogen source in organic form is chosen from the group consisting of a yeast extract, a corn steep liquor, and a combination thereof. More particularly preferably, the nitrogen source in organic form is a yeast extract. Preferably, the yeast extract is obtained from Saccharomyces cerevisiae.

The nitrogen source in organic form is added to the minimum medium comprising an inorganic nitrogen source. Preferably, the supply of nitrogen in organic form does not exceed 10% of the total nitrogen contained in the fermentation medium (inorganic and organic sources combined).

The inorganic nitrogen source in the minimum medium may be for example (NH₄)₂SO₄ or NH₄Cl.

The minimum medium may be supplemented with 0.5 to 3 g/l of yeast extract, preferably with 1 to 2 g/l of yeast extract. More particularly preferably, the minimum medium is supplemented with approximately 1 g/l of yeast extract. As it is used here, the term “approximately” refers to a value +/−20%, 10%, 5% or 2%.

The minimum medium may also be supplemented with 1 to 5 g/l of corn steep liquor, preferably 3 to 5 g/l and very particularly preferably with 4 g/l of corn steep liquor.

According to one embodiment, the method according to the invention makes it possible to increase the content of proteins in the biomass by at least 5% by total weight of the biomass, compared to the content of proteins in the biomass cultivated solely in minimum medium. The method according to the invention may make it possible to increase the content of proteins in the biomass by at least 6, 7, 8, 9 or 10% by total weight of the biomass, compared to the content of proteins in the biomass cultivated solely in minimum medium.

Preferably, the content of proteins in the biomass obtained by the method according to the invention is more than 45%, 50% or 55% by total weight of the biomass. According to another embodiment, the method according to the invention makes it possible to increase the content of carotenoids in the biomass by at least 0.05% by total weight of the biomass, compared to the content of carotenoids in the biomass cultivated solely in minimum medium. The method according to the invention may make it possible to increase the content of carotenoids in the biomass by at least 0.1 or 0.2% by total weight of the biomass, compared to the content of carotenoids in the biomass cultivated solely in minimum medium.

Preferably, the content of carotenoids in the biomass obtained by the method according to the invention is more than 0.35, 0.4 or 0.5% by total weight of the biomass.

According to another aspect, the present invention also relates to a method for heterotrophically culturing microalgae comprising:
a first step of culturing the microalgae in a minimum medium, and
a second culturing step in which a nitrogen source in organic form, chosen from the group consisting of yeast extract, corn steep liquor, and a combination thereof, is added to the minimum medium.
   The first step enables the growth of the microalgae and the second step prevents the accumulation of polysaccharide storage substances and makes it possible to enrich the biomass with proteins and carotenoids.

According to one embodiment, the microalga is of the Chlorella genus, preferably chosen from Chlorella sorokiniana, Chlorella vulgaris and Chorella kessleri, and is preferably Chlorella sorokiniana.

The nitrogen source in organic form added in the second culturing step is preferably yeast extract.

The present invention more particularly relates to a method for heterotrophically culturing said microalgae, especially Chlorella sorokiniana, comprising:
a first step of growing the microalgae in a minimum medium,
a second step in which yeast extract is added to the minimum medium.

According to one specific embodiment, the addition of a nitrogen source in organic form does not exceed 10% of the total nitrogen contained in the fermentation medium. In particular, the minimum medium may be supplemented with 0.5 to 3 g/l of yeast extract, preferably with 1 to 2 g/l of yeast extract.

Preferably, the second culturing step makes it possible to increase:
the content of carotenoids in the biomass by at least 0.05%, preferably by at least 0.1% by total weight of the biomass, compared to the content of carotenoids in the biomass cultivated solely in minimum medium, and/or
the content of proteins in the biomass by at least 5%, preferably by at least 10% by total weight of the biomass, compared to the content of proteins in the biomass cultivated solely in minimum medium.

According to one preferred mode, the content, in the biomass obtained,
of carotenoids is at least 0.35% by total weight of the biomass, preferably at least 0.4% by total weight of the biomass, and/or
of proteins is at least 45% by total weight of the biomass, preferably at least 50% by total weight of the biomass.

Optionally, and as will be demonstrated in the examples below, in the second step, glucose is supplied continuously at a value significantly below the glucose consumption capacity of said microalgae.

The embodiments described above relating to the method for carotenoid enrichment and protein enrichment of a microalgal biomass are also envisioned in this aspect.

The invention will be understood more clearly from the following examples which are intended to be illustrative and nonlimiting.

### EXAMPLE

### Production of C. sorokiniana—Addition of Yeast Extract

The strain used is Chlorella sorokiniana UTEX 1663.

Preculture:
600 ml of medium in a 2 l Erlenmeyer flask;
Composition of the medium:

| | | |
|---|---|---|
| Macro | Glucose | 20 |
| elements | K₂HPO₄•3H₂O | 0.7 |
| (g/l) | MgSO₄•7H₂O | 0.34 |
| | Citric acid | 1.0 |
| | Urea | 1.08 |
| | Na₂SO₄ | 0.2 |
| | Na₂CO₃ | 0.1 |
| | clerol FBA 3107 (antifoam) | 0.5 |
| Micro | Na₂EDTA | 10 |
| elements | CaCl₂•2H₂O | 80 |
| (mg/l) | FeSO₄•7H₂O | 40 |
| | MnSO₄•4H₂O | 0.41 |
| | CoSO₄•7H₂O | 0.24 |
| | CuSO₄•5H₂O | 0.24 |
| | ZnSO₄•7H₂O | 0.5 |
| | H₃BO₃ | 0.11 |
| | (NH₄)₆Mo₇O₂₇•4H₂O | 0.04 |

The pH is adjusted to 7 before sterilization by addition of 8N NaOH.

Incubation is carried out under the following conditions: duration: 72 h; temperature: 28° C.; stirring: 110 rpm (Infors Multitron incubator).

The preculture is then transferred to a 30 l Sartorius type fermenter.

Culture for Biomass Production:

The basic medium is identical to that of the preculture, but the urea is replaced by NH₄Cl:

| | | |
|---|---|---|
| Marco | Glucose | 20 |
| elements | K₂HPO₄•3H₂O | 0.7 |
| (g/l) | MgSO₄•7H₂O | 0.34 |
| | Citric acid | 1.0 |
| | NH₄Cl | 1.88 |
| | Na₂SO₄ | 0.2 |
| | clerol FBA 3107 (antifoam) | 0.5 |
| Micro | Na₂EDTA | 10 |
| elements | CaCl₂•2H₂O | 80 |
| (mg/l) | FeSO₄•7H₂O | 40 |
| | MnSO₄•4H₂O | 0.41 |
| | CoSO₄•7H₂O | 0.24 |
| | CuSO₄•5H₂O | 0.24 |
| | ZnSO₄•7H₂O | 0.5 |
| | H₃BO₃ | 0.11 |
| | (NH₄)₆Mo₇O₂₇•4H₂O | 0.04 |

Test 1: control; no nutritional supplement is added.

Test 2: 1 g/l of yeast extract is added.

The initial volume (Vi) of the fermenter is adjusted to 13.5 l after inoculation. It is finally brought to 16-20 l.

The parameters for carrying out the fermentation are as follows:

| Temperature | 28° | C. |
|---|---|---|
| pH | 6.5-6.8 by 28% w/w NH₃ | |
| pO₂ | >20% (maintained by stirring) | |
| Stirring | Minimum 300 rpm | |
| Air flow rate | 15 | l/min |

When the glucose supplied initially has been consumed, a medium similar to the initial medium is supplied in the form of a concentrated solution, containing especially 500 g/l of glucose.

The table below gives the composition of one liter of this concentrated solution:

| | | |
|---|---|---|
| Macro | Glucose | 500 |
| elements | K₂HPO₄•3H₂O | 34 |
| (g) | MgSO₄•7H₂O | 8.5 |
| | Citric acid | 25 |
| | Na₂SO₄ | 5.0 |
| Micro | Na₂EDTA | 250 |
| elements | CaCl₂•2H₂O | 2000 |
| (mg) | FeSO₄•7H₂O | 1000 |
| | MnSO₄•4H₂O | 10 |
| | CoSO₄•7H₂O | 6 |
| | CuSO₄•5H₂O | 6 |
| | ZnSO₄•7H₂O | 12 |
| | H₃BO₃ | 3 |
| | (NH₄)₆Mo₇O₂₇•4H₂O | 1 |

The concentrations of the elements other than the glucose have been determined such that they are in excess relative to the nutritional requirements of the strain.

This solution is supplied continuously at a rate lower than the glucose consumption capacity of the strain. In this way, the residual glucose content in the medium is kept at zero; that is to say that the growth of the strain is limited by the glucose availability (glucose-limiting conditions).

This rate is increased exponentially over time according to the following formula:

S=12. exp (0.07×t)

in which S=glucose supply rate (in g/h) and t=duration of fed batch mode (in h) Clerol FBA 3107 antifoam is added as required to avoid excessive foaming.

Results: Effect of Adding Yeast Extract

The content of proteins in the biomass obtained is evaluated by measuring the total nitrogen expressed by N 6.25.

| | | Duration | Biomass | % N | % |
|---|---|---|---|---|---|
| Test | Medium | (h) | (g/l) | 6.25 | carotenoids |
| 1 | Base | 72 | 75.7 | 40.0 | 0.3 |
| 2 | Base + 1 g/l of yeast | 71 | 76.3 | 50.2 | 0.4 |
| | extract | | | | |

These results show that supplying a nutritional supplement in the form of yeast extract makes it possible to obtain a high biomass concentration with a content of proteins of greater than 50%.

The content of carotenoids is also increased.

1-16. (canceled)

17. A method for carotenoid enrichment and protein enrichment of a heterotrophically cultivated microalgal biomass, comprising culturing said microalgal biomass in a minimum medium supplemented with a nitrogen source in organic form.

18. The method as claimed in claim 17, characterized in that the microalga is of the Chlorella genus.

19. The method as claimed in claim 17, characterized in that the microalga is selected from the group consisting of Chlorella sorokiniana, Chlorella vulgaris and Chorella kessleri.

20. The method as claimed in claim 19, characterized in that the microalga is Chlorella sorokiniana.

21. The method as claimed in claim 17, characterized in that the nitrogen source in organic form is selected from the group consisting of yeast extract, corn steep liquor, and a combination thereof.

22. The method as claimed in claim 21, characterized in that the nitrogen source in organic form is yeast extract.

23. The method as claimed in claim 21, characterized in that the nitrogen source in organic form is corn steep liquor.

24. The method as claimed in claim 17, characterized in that the addition of a nitrogen source in organic form does not exceed 10% of the total nitrogen contained in the fermentation medium.

25. The method as claimed in claim 17, characterized in that the minimum medium is supplemented with 0.5 to 3 g/l of yeast extract.

26. The method as claimed in claim 17, characterized in that the content of carotenoids in the biomass is increased by at least 0.05% of the total weight of the biomass, compared to the content of carotenoids in a biomass cultivated solely in minimum medium.

27. The method as claimed in claim 17, characterized in that the content of proteins in the biomass is increased by at least 5% of the total weight of the biomass, compared to the content of proteins in a biomass cultivated solely in minimum medium.

28. The method as claimed in claim 17, characterized in that the content of carotenoids in the biomass obtained is at least 0.35% by total weight of the biomass.

29. The method as claimed in claim 17, characterized in that the content of proteins in the biomass obtained is at least 45% by total weight of the biomass.

30. The method as claimed in claim 17, characterized in that it comprises:
a first culturing step of culturing the microalgae in a minimum medium, and
a second culturing step in which yeast extract or corn steep liquor is added to the minimum medium.

31. The method as claimed in claim 17, characterized in that it comprises:
a first culturing step of growing Chlorella sorokiniana in minimum medium, and
a second culturing step in which yeast extract is added to the minimum medium.

32. The method as claimed in claim 30, characterized in that, in the second step, glucose is supplied continuously at a value significantly below the glucose consumption capacity of said microalgae.

33. The method as claimed in claim 31, characterized in that, in the second step, glucose is supplied continuously at a value significantly below the glucose consumption capacity of said microalgae.
